SEQUENCE LISTING (3 sequences)

SEQ ID NO: 1 - DNA, 22 bp, Ubiquitin 5 of Arabidopisis thaliana
aaagaaggagttgaagcttgat

SEQ ID NO: 2 - DNA, 25 bp, Ubiquitin 11 of Arabidopisis thaliana
gccgactacgacatccagaaggagt

SEQ ID NO: 3 - DNA, 60 bp, Ubiquitin 11 of Arabidopsis thaliana
caacgtcaaggccaagatccaggataaggaaggtatccctccggaccagcagaggttgat

(19) United States
(12) Patent Application Publication
(10) Pub. No.: US 2003/0190663 A1
(43) Pub. Date: Oct. 9, 2003

(54) Novel assay for nucleic acid analysis

(75) Inventors: Li Yang, San Diego, CA (US); Xun Wang, Research Triangle Park, NC (US); Tong Zhu, Research Triangle Park, NC (US); Liang Shi, San Diego, CA (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 10/428,697

(22) Filed: May 2, 2003

Related U.S. Application Data
(62) Division of application No. 09/863,527, filed on May 14, 2001, now abandoned.
Application No. 09/863,527 is a continuation-in-part of application No. 09/565,214, filed on May 4, 2000, now abandoned.

(51) Int. Cl. C12Q 1/68

(52) U.S. Cl. 435/6

(57) ABSTRACT

Currently, three technologies are utilized for analysis of gene expression: hybridzation-based technologies, PCR-based technologies, and sequence-based technologies The present invention provides a method for analyzing the presence and/or amount of a specific nucleic acid using a solid support and a capture probe complementary to a region of a target nucleic acid, and polymerizing a labeled extension complementary to the target nucleic acid The invention provides a method of analysis of all types of nucleic acids, and can be used to study multiple genes in a single assay using different capture probes conjugated to different class of microspheres that can be mixed in any desired combination.

### RELATED APPLICATIONS

The present application is a continuation-in-part of U.S. patent application Ser. No. 09/565,214, filed May 4, 2000, by Yang et al., and entitled “NOVEL ASSAY FOR NUCLEIC ACID ANALYSIS,” which is hereby incorporated by reference herein in its entirety.

### FIELD OF THE INVENTION

The present invention relates to methods of analysis of nucleic acids. More specifically, the invention provides methods for analyzing the presence and amount of one or more specific nucleic acids using a solid support/capture probe system to capture a target nucleic acid, typically followed by polymerization of an extension complementary to the target nucleic acid.

### BACKGROUND OF THE INVENTION

Analysis of gene expression currently employs three primary technologies: hybridization-based techniques (northern blotting, subtraction cloning and DNA microarrays), PCR-based techniques (differential display) and sequence-based techniques (SAGE, MASS-spectrometry sequencing, ESTs). Among these approaches, northern blotting and microarrays are most broadly employed for gene expression studies, while other methods are applied more or less for the purpose of gene discovery, gene cloning, or library construction. The above technologies have a number of inherent problems. Northern blotting is a slow, laborious process that is not well suited for the evaluation of multiple samples or probes. As a screening tool, northern blotting is particularly unsuited. Hybridization-based technologies, such as gene chips, can be very expensive and can require a large amount of specialized equipment. In addition, gene chips require a long hybridization period, and are thus not suited for screening large numbers of different samples. Although a substantial number of genes are included on the chips, they can only be screened with a finite number of probes, and the chips are usually pre-made, preventing the user from flexibly designing and modifying a screening protocol directed to selected groups of genes or other nucleic acids of interest.

### SUMMARY OF THE INVENTION

Aspects of the present invention provide methods of analysis of a nucleic acid sample. In some embodiments, the method includes the steps of: providing a substrate including a solid support and a capture probe linked thereto, the capture probe having a sequence complementary to a first segment of a sequence of a single-stranded target nucleic acid; contacting the substrate with a nucleic acid sample, under conditions suitable for hybridization between the capture probe and the target nucleic acid, wherein upon the hybridization at least a second segment of the sequence of the target nucleic acid remains single stranded; exposing the substrate to conditions suitable for complementing at least a second segment of the target nucleic acid, wherein the complementing nucleic acid comprises nucleotides having a label capable of enhancing sensitivity of detection of the complementing nucleic acid.

In a preferred embodiment of the invention, an extension complementary to the second segment of the target nucleic acid may be polymerized, wherein the extension includes nucleotides having a label capable of enhancing sensitivity of detection of the extension; and analyzing the label to determine presence or absence of the target nucleic acid in the nucleic acid sample.

In a further embodiment of the invention, the substrate is exposed to conditions that allow hybridization with a probe molecule comprising one or more nucleotides having a modification or label capable of enhancing sensitivity of detection of the probe molecule, which is complementary to part or all of at least a second segment of the target nucleic acid.

According to this aspect of the invention, the analyzing step can include a quantitation of the label associated with the target nucleic acid. The solid support can be, for example, a microbead, a chromatography bead, an affinity bead, a gene chip, a membrane, a microtiter plate, a glass plate, a plastic plate, or the like. The solid support can be a fluorescent microbead, and can include one, two, or more fluorochromes; preferably, the different fluorochromes emit fluorescence at different wavelengths to indicate a fluorochrome identity of the microbead. The substrate can include a plurality of microbeads of at least two different classes, wherein the classes are based on fluorochrome identities of the microbeads within each class, and wherein the different classes of microbeads correspond to different target nucleic acids.

The methods of this aspect of the invention can further include the steps of: detecting the fluorescence of each of the different fluorochromes to determine the fluorochrome identity of the microbead; and correlating the analyzed label with the fluorochrome identity of the microbead. The analyzing step can include a quantitation of the label associated with the target nucleic acid. Likewise the microbead can be sorted based on its fluorochrome identity. The target nucleic acid can be, for example, mRNA, cRNA, viral RNA, synthetic RNA, cDNA, genomic DNA, viral DNA, plasmid DNA, synthetic DNA, a PCR product, or the like, and preferably can be derived from a plant, animal, virus or fungus.

The methods of this aspect of the invention can be used to identify a single nucleotide polymorphism in the target nucleic acid. In such embodiments, the nucleic acid can be derived from an organism and can be associated with a specific phenotype or trait of the organism. The extension can be polymerized by an enzyme, for example, a reverse transcriptase, a DNA polymerase, an RNA polymerase, Klenow fragment, or by a mutant form of any such enzyme. The label can be, for example, radionuclides, fluorescers, chemiluminescers, dyes, enzymes, enzyme substrates, enzyme cofactors, enzyme inhibitors, enzyme subunits, antigens, ligands, and metal ions; specifically, the label can be, for example, xanthine dyes, rhodamine dyes, naphthylamines, benzoxadiazoles, stilbenes, pyrenes, acridines, Cyanine 3, Cyanine 5, phycoerythrin, Alexa 532, fluorescein, TAMRA, tetramethyl rhodamine, fluorescent nucleotides, digoxigenin, biotin, or the like. The substrate can include a plurality of species of capture probes, and probes within each of the species can have a sequence distinct from the probes of every other of the plurality of species. In some embodiments, at least two of the plurality of species of capture probes can correspond to different segments of a single target nucleic acid, or the plurality of species of capture probes can correspond to different target nucleic acids.

Likewise, in some embodiments, the substrate can include more than 10 species of capture probes. The solid support can be, for example, a gene chip, a membrane, a glass plate, a plastic plate, or the like, and each of the species of capture probes can be linked to a discrete region of the solid support. Alternatively, each of a plurality of discrete regions of the solid support can have linked thereto probes of a plurality of species. In other embodiments, the substrate can include a plurality of solid support units, wherein the solid support units are, for example, a microbead, a chromatography bead, an affinity bead, a fluorescent bead, a radiolabeled bead, or the like. Each such solid support unit can have linked thereto only probes of one of the species. Alternatively, each solid support unit can have liked thereto probes of a plurality of species.

In some embodiments, the method can include the additional steps of: identifying solid support regions or units indicative of the presence of the target nucleic acid, based on the analyzing step; determining all species of capture probes linked to solid support regions or units of the identifying step; providing a second substrate, the second substrate including the probe species of the determining step, wherein the probe species are distinguishable from each other based on a discrete position of each species on a solid support including a plurality of the species, or based on presence of only a single species on each of a plurality of solid support units; contacting the second substrate with the nucleic acid sample, under conditions suitable for hybridization between a probe species and the target nucleic acid, wherein upon the hybridization, at least a second segment of the sequence of the target nucleic acid remains single stranded; exposing the substrate to conditions suitable for complementing at least a second segment of the target nucleic acid, wherein the complementing nucleic acid comprises nucleotides having a label capable of enhancing sensitivity of detection of the complementing nucleic acid.

In a preferred embodiment of the invention, an extension complementary to the second segment of the target nucleic acid may be polymerized, wherein the extension includes nucleotides having a label adapted to enhance sensitivity of detection of the extension; and analyzing the label to identify a probe species hybridized to the target nucleic acid.

In a further embodiment of the invention, the substrate is exposed to conditions that allow hybridization with a probe molecule comprising one or more nucleotides having a modification or label capable of enhancing sensitivity of detection of the probe molecule, which is complementary to part or all of at least a second segment of the target nucleic acid.

In another aspect of the invention, there are provided methods of screening an effect of a substance or changes in the environmental conditions on expression or regulation of a target nucleic acid in a biological system, including the steps of: treating the biological system with the substance or subjecting it to changed environmental conditions; extracting a nucleic acid sample from the biological system; providing a substrate including a solid support and a capture probe linked thereto, the capture probe having a sequence complementary to a first segment of a sequence of a single-stranded target nucleic acid; contacting the substrate with the nucleic acid sample extracted from the biological system, under conditions suitable for hybridization between the capture probe and the target nucleic acid, wherein upon the hybridization at least a second segment of the sequence of the target nucleic acid remains single stranded; exposing the substrate to conditions suitable for complementing at least a second segment of the target nucleic acid, wherein the complementing nucleic acid comprises nucleotides having a label capable of enhancing sensitivity of detection of the complementing nucleic acid.

In a preferred embodiment of the invention, an extension complementary to the second segment of the target nucleic acid is polymerized, wherein the extension includes nucleotides having a label capable of enhancing sensitivity of detection of the extension; analyzing the label to determine presence or absence of the target nucleic acid in the nucleic acid sample; and determining the effect of the substance on expression or regulation of the target nucleic acid in the biological system.

In a further embodiment of the invention, the substrate is exposed to conditions that allow hybridization with a probe molecule comprising one or more nucleotides having a modification or label capable of enhancing sensitivity of detection of the probe molecule, which is complementary to part or all of at least a second segment of the target nucleic acid.

The biological system can be, for example, a cell or cell culture, a tissue, an organ, an individual organism, a population of individuals of a single taxon, a combination of cells, tissues, organs, or individuals of different taxa, or the like. The system preferably includes a plant, an animal, a fungus, or a part of a plant, animal, or fungus. The substance can include one or more components, for example, an organic substance, an ion, a mineral, a vitamin, a hormone, a gas, a virus, a bacterium, a fungus, or the like. The environmental conditions may be altered by changing salt concentration, pH, temperature, population density, or other factors that have the potential to influence the physiological state of the biological system upon being subjected to those changes.

The effect of the substance or the changes in the environmental conditions on expression or regulation of the target nucleic acid may include completely suppressing the expression, reducing the rate of expression, or, in other embodiments, increasing the rate of expression.

Another aspect of the invention provides systems of gene expression analysis. In a preferred embodiment, the system includes a microbead having at least two different fluorochromes, and further includes at least one capture probe linked to the microbead, the capture probe having a sequence complementary to a first segment of a sequence of a target nucleic acid, the system also including a labeled probe complementary to at least second segment of the sequence of the target nucleic acid, wherein the labeled probe includes a label capable of enhancing sensitivity of detection thereof. The labeled probe can be a product of nucleic acid polymerization within the complex, using the second segment as a template therefor. The labeled probe can include a first region complementary to the second segment of the target nucleic acid and a second region capable of interacting faith a signal enhancer. The second region can be branched in structure, ha-.ing a plurality of ends, with at least two of the ends being capable of interacting with a signal enhancer. The signal enhancer can be, for example, a labeled probe, radionuclides, fluorescers, chemiluminescers, dyes, enzymes, enzyme substrates, enzyme cofactors, enzyme inhibitors, enzyme subunits, antigens, ligands, metal ions, or the like.

A further aspect of the invention provides a diagnostic kit suitable for diagnosis of a particular physiological state of an organism, including a solid support and a capture probe linked to the solid support, wherein the capture probe is complementary to a first segment of a target nucleic acid associated with the physiological state. The kit can further include a probe capable of hybridizing to at least a second segment of the target nucleic acid; the probe can include a label capable of enhancing sensitivity of detection thereof. The kit can further include components necessary for extension of a probe or one or more labeled probe molecules complementary to at least a second segment of the target nucleic acid.

In another aspect, the invention provides methods of marker assisted breeding including the steps of: providing a substrate including a solid support and a capture probe linked thereto, the capture probe having a sequence complementary to a first segment of a sequence of a target nucleic acid, wherein the target nucleic acid is correlated with a trait of interest in a breeding program; contacting the substrate with a nucleic acid sample from an individual or population in the breeding program, under conditions suitable for hybridization between the capture probe and the target nucleic acid; probing a second segment of the target nucleic acid to detect presence or absence of the target nucleic acid; and determining desirability of the individual or population for the breeding program, based on the presence or absence of the target nucleic acid. The probing step preferably includes polymerization of a probe using the second segment as a template therefor. The solid support can be a microbead having at least two different fluorochromes. The trait can be correlated with a plurality of target nucleic acids, and wherein the substrate includes capture probes complementary to at least two of the target nucleic acids. The method can be used to screen candidates for breeding, and/or to screen progeny of the breeding program for end use or for subsequent breeding steps.

In yet another aspect, the invention provides methods of determining effectiveness of a capture probe, including the steps of: providing a substrate including a solid support and a capture probe linked thereto, the capture probe having a sequence complementary to a first segment of a sequence of a single-stranded target nucleic acid; contacting the substrate with a nucleic acid sample, under conditions suitable for hybridization between the capture probe and the target nucleic acid, wherein upon the hybridization at least a second segment of the sequence of the target nucleic acid remains single stranded; exposing the substrate to conditions suitable for complementing at least a second segment of the target nucleic acid, wherein the complementing nucleic acid comprises nucleotides having a label capable of enhancing sensitivity of detection of the complementing nucleic acid

In a preferred embodiment of the invention, an extension complementary to the second segment of the target nucleic acid may be polymerized, wherein the extension includes nucleotides having a label capable of enhancing sensitivity of detection of the extension; and analyzing the label quantitatively to determine effectiveness of the capture probe in capturing the target nucleic acid.

In a further embodiment of the invention, the substrate is exposed to conditions that allow hybridization with a probe molecule comprising one or more nucleotides having a modification or label capable of enhancing sensitivity of detection of the probe molecule, which is complementary to part or all of at least a second segment of the target nucleic acid.

### DETAILED DESCRIPTION OF THE INVENTION

Disclosed herein is a process for nucleic acid analysis that is inexpensive, fast, flexible, and applicable to high-throughput technology. The method typically employs a substrate, which in a preferred embodiment includes a plurality of microbeads, each bead belonging to a “class” based on the fluorochromes associated with it. The fluorochromes allow for identification of each bead class. Each separate class of beads may be associated with a particular capture probe or a group of capture probes. The capture probe may be a single-stranded nucleic acid molecule that corresponds to the target nucleic acid of interest.

The preferred length of the capture probe is between 22 and 25 bases. However, also smaller and larger sized capture probes may be suitably employed in the method according to the invention. On the lower side, a size for the capture probe of between 15 and 18 bases is preferred, while on the upper side the preferred size is between 60 and 150 bases In order to avoid cross-hybridization with genes other than the gene of interest, the capture probe is designed such that it is complementary to a unique sequence within the gene of interest. Mismatches within the capture probe sequence may be allowed as long as they do not interfere with the specificity of the capture probe. With increasing length of the capture probe, the amount of acceptable mismatches is also increasing.

The capture probe may be complementary to essentially any region within the gene of interest, as long as this region is unique amongst all the genes tested. The preferred region is a region comprising approximately 1000 bases from the 3′-end of the gene of interest, preferably approximately 800 bases and even more preferably approximately 600 bases from the 3′-end of the gene of interest.

In practice, a nucleic acid sample is added to the substrate, and may be denatured either before or after combination with the substrate. The single stranded target nucleic acid binds to the corresponding capture probe. The capture probe is designed such that it binds to a segment of the target nucleic acid, leaving at least one other segment of the target single stranded. The mixture is exposed to conditions that allow for complementing at least a second segment of the target nucleic acid, wherein the complementing nucleic acid comprises nucleotides having a label capable of enhancing sensitivity of detection of the complementing nucleic acid. In a preferred embodiment of the invention, an extension is polymerized that is complementary to at least one segment of the target not hybridized to the capture probe. The lower size limit of the complementing nucleic acid is preferably between 12 and 15 nucleotides whereas on the upper side the preferred range is between 150 and 200 nucleotides.

Especially preferred is a size range of between 18 and 25 nucleotides.

During polymerization, a label may be incorporated into the extension. This may be achieved, for example, by offering one or more modified or labeled nucleotides in the polymerization process. The label can be, for example, radionuclides, fluorescers, chemiluminescers, dyes, enzymes, enzyme substrates, enzyme cofactors, enzyme inhibitors, enzyme subunits, antigens, ligands, and metal ions; specifically, the label can be, for example, xanthine dyes, rhodamine dyes, naphthylamines, benzoxadiazoles, stilbenes, pyrenes, acridines, Cyanine 3, Cyanine 5, phycoerythrin, Alexa 532, fluorescein, TAMRA, tetramethyl rhodamine, fluorescent nucleotides, digoxigenin, biotin, or the like.

The overall ratio of modified/labeled vs unmodified/unlabeled nucleotides in the reaction mixture is preferably between about 1:7 and 1:2, more preferably between 1:5 and 1:2, most preferably the ration is about 1:3. Preferred are nucleotides incorporating modified bases, such as, for example, biotinylated or fluoresceinated nucleotides. In particular, at least one of the 4 bases A, T, C and G may be offered in a labeled form. For example, biotin-16-dUTP or, alternatively, biotin-14-dCTP may preferably be incorporated into the extended DNA during the polymerization reaction. The resulting extension may then contain incorporated therein between about one modified or labeled nucleotide of every 10 to 50, preferably every 15 to 35, more preferably every 20 to 25 nucleotides. This label then may be analyzed, qualitatively and/or quantitatively, to determine the presence and/or relative abundance of the target nucleic acid.

In a further embodiment of the invention, the substrate is exposed to conditions that allow hybridization with a probe molecule comprising one or more nucleotides having a modification or label capable of enhancing sensitivity of detection of the probe molecule, which is complementary to part or all of at least a second segment of the target nucleic acid. The label may reside with any one of the four bases A, T, G or C. For example, biotin-16-dUTP or, alternatively, biotin-14-dCTP may preferably be incorporated into the extended DNA during the polymerization reaction. The probe molecules may be end-labeled at the 5′- and/or 3′-end and may contain additional modified or labeled nucleotides along the nucleotide stretch, such as, for example, radiolabeled, biotinylated or fluoresceinated nucleotides. The resulting probe molecule may then contain incorporated therein between about one modified or labeled nucleotide of every 10 to 50, preferably every 15 to 35, more preferably every 20 to 25 nucleotides.

The sensitivity of detection may be further increased by adding two or more labeled probe molecules to the reaction mixture, which are complementary to different parts of at least a second segment of the gene of interest, which is not bound to the capture probe and still accessible for hybridization with the additional probe molecules. Preferred is a number of labeled probe molecules in a range of between about I and 10, preferably of between about 1 and 5 and most preferably of between about 1 and 3 probe molecules.

The invention is particularly well suited for multiplex analysis of gene expression.

Since large numbers of classes of microbeads can be used simultaneously, each directed either to a single target nucleic acid or a pool of selected target nucleic acids, it is possible by the method to detect, qualitatively or quantitatively, the expression or presence of hundreds or thousands of genes in one experiment. Using embodiments of this method, it is possible to assess the effects on gene expression of chemicals, pathogens, stress conditions and other environmental perturbations, developmental stages, and the like. It is also possible, using embodiments of the method, to screen individuals or populations for marker sequences associated with desirable or undesirable traits or phenotypes, greatly enhancing the efficacy of marker-assisted breeding of plants, animals, or other organisms of economic or research significance. Further, the methods disclosed herein are useful for high throughput screening of drugs and other substances for their effects on expression of target nucleic acids.

Because of the incorporation of label via a strand extension reaction, target nucleic acids can be detected in extremely small quantities, significantly increasing the sensitivity and range of detection of the target. Within a significant portion of this range, incorporated label can be used to assess quantitative dynamics of gene expression, to distinguish between heterozygous and homozygous individuals for several loci simultaneously, and to identify populations with a desirably high or low frequency of one or more alleles or markers associated with a phenotype or trait.

Certain embodiments of the invention use the commercially available Luminex microfluidics analyzer and color-coded microspheres (Luminex Corporation, Austin, Tex.) to provide a rapid, sensitive, and multiplexed assay for gene expression. Oligonucleotide capture probes derived from target genes are synthesized and immobilized to microspheres via a simple chemical coupling reaction. The microfluidics/fluorescence technology makes it possible to distinguish numerous different classes of microspheres.

The multiplex potential of the method is a function of the number of detectably different microsphere classes. For example, using 25 different microbead classes, with each class having a unique capture probe linked thereto, 25 different targets can be analyzed in one experiment. However, by pooling larger numbers of capture probes or by using larger numbers of classes of beads, is it possible to screen hundreds or thousands of target nucleic acids in a single experiment. For example, by using 100 different microbead classes, and by linking a pool of 20 different selected capture probes to each class of beads, an initial capture/strand extension reaction can detect up to 2000 target sequences. This may be followed by a second capture/strand extension reaction using, for example, 20 different microbead classes, each having only one kind of capture probe (from the pool of 20 probes initially linked to a single microbead class), allowing precise determination of all target nucleic acids actually captured by one microbead class in the first reaction.

As an alternative to pooled screening using microbeads, multiple capture probes can be linked to a particular discrete region of a non-bead solid support, such as a gene chip, a membrane, a glass slide, or the like, and a first round of hybridizations can be performed to identify which of the discrete regions may have capture probes that correspond to target nucleic acids in the sample. This is then followed by one or more subsequent rounds of hybridizations to different solid supports having subsets of the pooled capture probes, in order to identify which individual capture probe(s) in the original pool hybridized to the target nucleic acid in the sample.

In this pooled screening process, labeling of the target nucleic acid can be achieved through various means including, for example, ill situ strand extension incorporating labeled nucleotides, or hybridization with an unbound labeled probe complementary to a region of the target nucleic acid. Other techniques of pooling probes and then identifying individual targets can also be used in accordance with the method; various such techniques are known in the art and their application to the novel multiplex method will be evident to those of skill in the art.

Based on the fluorescence signature of each class of fluorescent microspheres, the microfluidics analyzer accurately distinguishes each microsphere class from every other class, and measures the total fluorescence at the bead's surface for each class to quantify the amount of labeled target, such as, for example, RNA or cDNA, specifically associated with the beads. To study multiple genes in a single assay, different capture probes representing each gene are conjugated to different classes of microspheres, and microspheres coupled with specific probes of the gene of interest are mixed in any desired combination. The fluorescence identity of each bead therefore correlates with its unique capture probe or combination of capture probes, and also correlates with its unique target or combination of targets. Thus, the assay is highly flexible, allowing easy addition or reduction of the numbers of genes for analysis. Other advantages of this method include high affordability, rapid processing and high throughput format. The method is very well suited for diagnostic detection of clinical samples and for identification of marker genes in crops, breeding, and screening.

This invention, like northern blotting and gene chips, is especially usefull for gene expression analysis. The advantages of this invention for gene expression analysis compared to the classical northern blotting and modem gene chips are discussed below.

Although invented over twenty years ago, northern blotting still has not lost its importance, and is often used to confirm differences detected in transcript expression. However, the traditional northern blotting method may require a week or more to obtain results, and one can only study a few samples each time. In addition, the power of northern blotting can be highly dependent on the quality of the RNA used. The present invention provides an alternative to northern blotting that takes about one hour per assay and can easily analyze hundreds of samples and multiple genes per experiment. Typically, northern blotting only analyzes one gene each time.

Laborious and time-consuming processes such as electrophoresis, staining, and washing are necessary in northern blotting, making the development of a high throughput use of northern blotting unlikely, if not impossible. The simple process of the present invention is very easily adaptable for a high throughput format. The materials cost of a typical single assay for the present invention is roughly comparable to the materials cost of a single northern blot. However, because multiple genes can be examined in a single assay of the present invention, the cost is significantly lower per data point than the cost of northern blotting. Taking labor cost into account, the relative cost of the novel method per data point is still lower in comparison to northern blotting. Employing the present invention, I to 10 μg of total RNA is typically sufficient for obtaining detectable signal for abundantly and moderately expressed genes. Northern blots usually require 5-30 μg of total RNA.

The introduction of gene chips advanced the study of gene expression profiles and genomic compositions tremendously. The technology involves attaching probes such as oligonucleotides derived from ESTs, PCR products or cloned cDNAs to the surface of nylon filters, glass slides or silicon chips at high density. To determine gene expression level, labeled cDNAs are hybridized to the DNA or oligonucleotides on the arrays and the hybridized signals are scanned, and measured via fluorescent probes on the gene-captured sites. The Affymetrix gene chip allows detection of about 7000 genes in one array—equal to the complete genome of yeast. Gene chips are thus a powerful tool for genomic profiling. However, limitations do exist for gene chips when using them for certain applications. For example, gene chips are not suited for studying small numbers of genes with an extremely large number of samples. For clinical diagnosis, drug screening, and marker gene identification in any screening system, it is often necessary to detect several specific genes from large numbers of samples. On the other hand, the present invention serves this purpose very well. It is excellent for diagnostic and screening applications.

Usually, the position of each gene in a gene chip is fixed. Customers do not have much freedom to select genes of their own interest under most circumstances. Having a custerized array for gene chip technology is extremely difficult in practice. For example, if positions of genes or compositions of genes are changed in a gene chip, the relevant software must be rewritten accordingly, which requires a tremendous amount of work from both the computer specialist and the biologist. In contrast, the present invention is very flexible for creating customized arrays. In a preferred embodiment, microspheres coupled with specific probes are easily mixed in any desired combinations for the detection of any gene of interest.

The Affymetrix gene chip technology uses expensive pre-made gene chips are also requires expensive instruments. The enormous amount of data necessary for accurate analysis must be analyzed by costly software. Making self-designed cDNA microarrays, as an alternative to buying pre-made Affymetrix gene chips, requires an expensive spotter and a high quality scanner. Most laboratories cannot afford the gene chips or the machinery used to analyze and/or make them. The low cost of the present invention for gene expression analysis is thus particularly attractive. While a microfluidics analyzer used to analyze the fluorescent microspheres in a preferred embodiment is relatively costly, the microspheres needed for analyses are very inexpensive per data point. Overall, the expense of the method of the invention is much lower per experiment than with gene chip technology.

The hybridization step for gene chips requires incubation for at least 16 hours, and cDNA chips require incubation for 4 hours. In the new method, the hybridization incubation typically lasts about 10 to 30 minutes. Therefore, a great deal of time is saved utilizing the invention to study gene expression analysis. Due to its high cost and long hybridization times, gene chip technology is not suited for screening large quantities of samples. In contrast, the present invention is much faster, less expensive, and more flexible than gene chips for high throughput screening of large numbers of samples.

The present invention contemplates a method for analysis of a nucleic acid sample using a solid support that, in a preferred embodiment, is a plurality of microbeads, each bead belonging to a “class” based on the fluorochrome(s) associated with it. This allows for identification of the bead class. Each separate class of beads has a particular species of capture probe attached to it. The capture probe is a single-stranded nucleic acid molecule that corresponds to the nucleic acid of interest to be detected or quantitated. A nucleic acid sample is added to the substrate and the target nucleic acid binds to the capture probe. The substrate with bound target is exposed to conditions that allow an extension to be polymerized complementary to a single-stranded segment of the target nucleic acid, such that a label is incorporated into the extension. This label is then analyzed to determine the presence or absence, or to quantitate the amount of the target nucleic acid.

The solid support may be, for example, a microbead, a chromatography bead, an affinity bead, a gene chip, a membrane, a microtiter plate, a glass plate or a plastic plate. The color-coded microspheres are a preferred embodiment and are a particularly advantageous solid support because they can be used with a microfluidics analyzer to identify specific microbeads that correspond to specific capture probes. Luminex beads typically correspond to a particular signature of two fluorochromes that can be easily identified. This allows, for multiple analyses at the same time.

The Luminex microbeads are extensively discussed in PCT Application No. PCT/US99/01315, filed Jan. 22, 1999 and published Jul. 29, 1999 as WO 99/37814. Briefly, the microbeads are microparticles that incorporate polymeric nanoparticles stained with one or more fluorescent dyes. All of the nanoparticles in a given population are dyed with the same concentration of a dye, and by incorporating a known quantity of these nanoparticles into the microsphere, along with known quantities of other nanoparticles stained with different dyes, a multifluorescent microsphere results. By varying the quantity and ratio of different populations of nanoparticles it is possible to establish and distinguish a large number of discrete populations of microspheres with unique emission spectra. The fluorescent dyes used are of the general class known as cyanine dyes, with emission wavelengths between 550 nm and 900 nm. These dyes may contain methine groups; the number of methine groups influences the spectral properties of the dye. The monomethine dyes that are pyridines typically have a blue to blue-green fluorescence emission, while quinolines have a green to yellow-green fluorescence emission. The trimethine dye analogs are substantially shifted toward red wavelengths, and the pentamethine dyes are shifted even further, often exhibiting infrared fluorescence emission. However, any dye compatible with the composition of the beads can be used.

When a number of different microbeads are used in the same assay in the present invention, it is preferable that the dyes have the same or overlapping excitation spectra, but possess distinguishable emission spectra. Multiple classes or populations of particles can be produced from just two dyes. The ratio of nanoparticle populations with red/orange dyes is altered by an adequate increment in proportion so that the obtained ratio does not optically overlap with the former ratio. In this way a large number of differently fluorescing microbead classes are produced.

When differentiation between the two dyes is accomplished by visual inspection, the two dyes preferably have emission wavelengths of perceptibly different colors to enhance visual discrimination. When it is desirable to differentiate between the two dyes using instrumental methods, a variety of filters and diffraction gratings allow the respective emission maxima to be independently detected. In a preferred embodiment, a microfluidics analyzer is used to distinguish the fluorescent microbeads. As an alternative to the use of a microfluidics analyzer, various embodiments of the invention are also suitable for use with a fluorescence-activated cell sorter, wherein the different classes of beads in a mixture can be physically separated from each other based on the fluorochrome identity of each class of bead, and the target nucleic acid and/or label associated therewith can be qualitatively or quantitatively determined for each sorted pool containing beads of a particular class.

In a preferred embodiment, the substrate may advantageously include a plurality of microbeads of at least two different classes to allow for separate identification of each class. The substrate may also include, for example, a plurality of chromatography beads or affinity beads, or a gene chip, a membrane, or a variety,of plates—microtiter, glass, or plastic. The invention thus contemplates the use of any solid support to which a capture probe can be linked.

The target nucleic acid as well as the capture probe may be any type of single-stranded nucleic acid. Double-stranded nucleic acids may also be processed (or denatured) in such a way as to produce nucleic acids that are single-stranded at least in some segments thereof, for at least a short time, that can then be linked to the solid support or used as a sample.

Examples of nucleic acids include but are not limited to: mRNA, cRNA, viral RNA, synthetic RNA, cDNA, genomic DNA, viral DNA, plasmid DNA, synthetic DNA, or a PCR product.

The nucleic acid may be derived from a plant, animal, fungus, virus or microorganism. For analysis of clinical samples, the nucleic acid advantageously may be derived from a human cell, tissue, or organ.

The target nucleic acid may be associated with a particular phenotype, disease state, or trait of the organism. This is particularly useful in the identification and quanitation of infection in a human, and identification of cancer-particularly residual cancer after treatment. This method is useful for screening a wide variety of diseases, genetic traits, risk factors, and, in the research setting, for identifying genes, polymorphisms, mutations, alleles, and the presence of foreign DNA. In the field of plant breeding and research, the method is useful for identifying or quantitating marker genes or sequences that may be associated with desirable or undesirable properties of crops, and can also be applied to other biological organisms. The invention is also useful for screening the effects of large numbers of candidate compounds on the expression of certain target genes.

The method is useful in association with many of the techniques already being used for quantitation of nucleic acids. For example, a battery of probes can be tested simultaneously to identify particularly efficient or inefficient probes, before use of such probes in microarrays, gene chips, northern blots, or other applications.

In accordance with the method, a capture probe is attached to a solid support. An example is the attachment of nucleic acid to microspheres using carbodiimide coupling. In this procedure, the polymeric particles have pendant carboxyl groups on the outer surfaces. The particles are composed of a poly(styrene-comethacrylic acid) (90:10 molar ratio). A sample of the polymeric particle is mixed with the carbodiimide, and amino-substituted oligonucleotides in acidic buffers; incubation is then continued for about 1 hour. The reaction mixtures are centrifuged, the supernatant discarded, and the pellets washed with one or more detergent solutions, and then resuspended in an acidic solution.

Covalent attachment to a variety of types of microbeads is accomplished using similar coupling methods, which are known in the art. Attachment to membranes, microtiter plates, glass, and plastic plates involves such processes as UV crosslinking, drying, heat, and treatment with NaOH. Coupling and crosslinking methods for attaching a nucleic acid probe to a solid support are known in the art; the most appropriate technique for a given application will be evident to those of skill in the art. For example, a plate can be coated with agarose containing streptavidin, and biotinylated oligonucleotides can be immobilized on the plate.

As an alternative, oligonucleotides can be attached to a solid support through solid phase synthesis thereon. Likewise, nucleic acids such as cDNAs can be attached to polylysine-treated glass slides.

The capture probe is designed such that it will bind to a portion of a target nuclear acid, leaving another portion of the target single-stranded. The single-stranded portion is the used as a template for strand extension during the polymerization step. The extension step involves the polymerization of nucleic acid using an enzyme and advantageously incorporating a label into the newly synthesized nucleic acid. A variety of enzymes can be used for this purpose, as will be appreciated by those of skill in the art. Examples of suitable enzymes include, but are not limited to: reverse transcriptase, DNA polymerase, RNA polymerase, fragments of these enzymes, such as Klenow fragment, and mutated enzymes that retain their nucleic acid polymerizing activity, but that can also incorporate modified nucleotides, such as, for example, biotinylated or fluoresceinated nucleotides.

The label that is incorporated into the polymerized nucleic acid may be selected based on the application. Examples of such labels include radionuclides, fluorescers, chemiluminescers, dyes, enzymes, enzyme substrates, enzyme cofactors, enzyme inhibitors, enzyme subunits, antigens, ligands, and metal ions, particularly: xanthine dyes, rhodamine dyes, naphthylamines, benzoxadiazoles, stilbenes, pyrenes, acridines, Cyanine 3, Cyanine 5, phycoerythrin, Alexa 532, fluorescein, TAMRA, tetramethyl rhodamine, fluorescent nucleotides, digoxigenin, and biotin. Likewise, in some embodiments, the nucleic acid can be labeled using intercalating dyes such as, for example, YOYO, TOTO, Picogreen, ethidium bromide, and the like.

A particularly advantageous embodiment of the method is its use in identifying a large number of different target nucleic acids within the same sample. This is accomplished by separate identification of each specific solid support unit, such as, for example, a microbead, that has a specific capture probe associated with it. In preferred embodiments, attachment of a specific capture probe to a microbead having a specific fluorochrome identity allows for such identification. Thus the specific capture probe can be identified by the bead “color.” Alternative embodiments can employ any of numerous other solid supports, such as, for example, a microtiter plate, a gene chip, a chromatography bead, and the like. Some embodiments may employ two or more capture probes corresponding to different segments of the same target nucleic acid, with the capture probes for any given target being preferably coupled to the same solid support unit.

The analysis step of the method is carried out in accordance with the label used. For high throughput screening, the fluorochromes on the beads can be identified and quantitated using a microfluidics analyzer by identifying the fluorochrome that corresponds to a specific capture probe. The green label can also be identified and quantitated in this way, and corresponds to a positive result. The amount of label can be analyzed, depending on the tape of label used. For example, if the label is biotin, it can be detected by the addition of phycoerythrin conjugated streptavidin. In fact, a detection method that includes an amplification step is particularly advantageous.

Several preferred embodiments for the analysis of gene expression in accordance With the invention are described using the following examples for illustration:

### EXAMPLES

Examples are provided below to illustrate different aspects and embodiments of the present invention. These examples are not intended in any way to limit the disclosed invention. Rather, they illustrate some of the methodology of the present invention.

### Example 1

Detecting Gene Expression at the RNA Level

A gene transcript can be detected directly from total RNA using capture probes coupled with microspheres. In this specific approach, capture probes are anti-sense oligonucleotide molecules corresponding to a first region of target RNAs. The capture probes are coupled with microspheres. Gene-representing targets are RNA transcripts, and label is added by extending a complementary strand along a second region of target RNAs.

A unique sequence of 22 bases complementary to a region close to the 3′-end of a target nucleic acid is chosen as a capture probe oligonucleotide. The capture probe oligonucleotide is synthesized with 5′-amino uni-linker (Oligos Etc., Seattle, Wash.) and then covalently linked to carboxylated fluorochrome microspheres following the classical carbodiimide coupling procedure (materials available from Sigma, St. Louis, Mo.).

Total RNA extracted from samples is fragmented and then denatured by incubation at 100° C. for 10 min. in a hybridization buffer of 1× TMAC. Microspheres coupled with capture probes are then added to the denatured RNA and incubated at 55° C. for 10 min. Target genes are hybridized selectively to their probes on microspheres and thereby immobilized. Strand extension, using the single-stranded region of the captured RNA as a template, is carried out by addition of a reverse transcriptase capable of incorporating labeled or modified nucleotides. The strand extension reaction follows conventional protocols, and is typically conducted at about 45° C. to 60° C. in the butter supplied with the enzyme. Labeled nucleotides are thus incorporated into the extending strand, creating a detectable complex of fluorescent bead/capture probe/target RNA/labeled extended strand. The mixture is passed through a microfluidics analyzer, and presence of certain target RNAs is indicated by presence of label on beads having a selected fluorescence identity. This protocol allows detection of target sequences in the femtomole range.

### Example 2

Detecting Gene Expression at the cDNA Level

Instead of capturing RNA sequences directly as described above, gene expression can also be analyzed from cDNA. In this method, capture probes are sense sequence oligonucleotides, gene-representing targets are cDNAs and label is incorporated either into the cDNA or into an extended second-strand cDNA, or both.

Total RNA extracted from a tissue or cell line is subjected to reverse transcription. Generally, 10 μg of total RNA is used per assay. Poly-(dT)₂₀ that contains biotin at the 5′-end serves as primer in the reaction. Sensitivity of the assay is enhanced by incorporation of biotinylated deoxynucleotide, biotin-16-dUTP (Roche Diagnostics Corp., Indianapolis, Ind.), into newly synthesized cDNA. As an alternative, primers of Poly(dT)₂₀ containing more than one molecule of biotin can be used to increase sensitivity of this assay.

An alternative labeling method is to use Cy3 labeled poly-(dT)₂₀ as a primer. Cy3-dUTP is added to the reaction and incorporated into cDNA during the reverse transcription. In many cases, Cy3-dUTP is incorporated more efficiently into cDNA than biotinylated deoxynucleotide. Again, a unique sequence of 22 bases close to the 3′-end of the gene of interest is selected as the capture probe. The capture probe oligonucleotides are synthesized with 5′-end amino uni-linker and subsequently coupled to carboxylated microspheres by the carbodiimide coupling method.

The labeled cDNA is denatured by incubation at 100° C. for 10 min in 1× TMAC buffer. Microspheres coupled with probes are added to the denatured cDNA and incubated at 55° C. for 10 min. Target genes with complementary sequence to the capture probe specifically associate with their corresponding microspheres during incubation. In the final step, second strand cDNA is synthesized, using the captured target cDNA as a template. Cy3-dUTP is incorporated into the second strand cDNA, and the reaction mix can be analyzed directly on a microfluidics analyzer.

### Example 3

Detecting Gene Expression in Arabidopsis by Second Strand cDNA Extension

Extending the second strand of cDNA on beads was conducted to assay gene expression in Arabidopsis. Using this approach, the detection of UBQ5 (Ubiquitin 5) and UBQ11 (Ubiquitin 11) from various amounts of Arabidopsis total RNA was performed. UBQ5 and UBQ11 are constitutively and abundantly expressed genes in Arabidopsis. A linear relationship between signal and the amount of RNA used was observed in this assay. In a separate multiplex assay, three defensive genes and UBQ5 (UBQ11) were detected simultaneously. These results are discussed later in this section.

Total RNA extracted from Arabidopsis was used as a template for reverse transcription and Poly-(dT)₂₀ was used as a primer. In this experiment, cDNA was synthesized without any labeling. Capture probes were designed as described above, comprising a unique sequence of 22, 25 and 60 bases, respectively, close to the 3′-end of the UBQ gene exhibiting the following nucleotide sequence:

UBQ5: X*aaagaaggagttgaagcttgat

UBQ11: X*gccgactacgacatccagaaggagt

UBQ11: X*caacg tcaaggccaa gatccaggat aaggaaggta tccctccgga ccagcagagg ttgat

and coupled to microspheres. Target cDNAs were then hybridized to the capture probes on microspheres. The microspheres were then centrifuged and the supernatant was removed. Following resuspension of the microspheres in DNA extension buffer, E. coli DNA polymerase I (Gibco BRL, Rockville, Md.) was added to the mix to extend the second strand cDNA using the capture probe as a primer and the first strand of cDNA as a template. For labeling, biotin-16-dUTP (Boehringer Mannheim) was incorporated into the extended DNA during the reaction. Alternatively, biotin-14-dCTP (Gibco BRL) was incorporated during the synthesis. Other polymerases such as Klenow fragment (Gibco BRL) and Platinum Taq polymerase (Gibco BRL) were also tested. Among the enzymes tested, E. coli DNA polymerase I performed the best for the extension under the conditions used.

X* in the above sequences is a uni-linker, which is added to the 5′-end during oligo synthesis to covalently link the capture probe to carboxylated fluorochrome microspheres as described in Example 1.

After the second strand DNA extension reaction, the microspheres were centrifuged again to remove the supernatant containing free biotin-16-dUTP. The microspheres were resuspended in hybridization buffer, and phycoerytlrin conjugated to streptavidin was added to the solution and incubated for 5 min to assure the binding to the incorporated biotin-16-dUTP.

UBQ5 and UBQ11, respectively, may be detected from 1 μg, 3 μg and 8 μg of total RNA extracted from wild type Arabidopsis leaves (see results f6r UBQ5 in table 1). A strong linear relationship was observed between the signal and the amount of RNA sample used. This result validated the application of the assay for gene expression analysis. It indicated that the method is sensitive enough to detect a high copy gene like UBQ5 and UBQ11, respectively. Meaningful signal was obtained from samples containing only 1 μg of total RNA; useful results thus can be obtained if moderately expressed genes are examined by this approach. For rarely expressed genes whose expression level is 100 times lower than that of UBQ5 and UBQ11, respectively, a larger initial amount of RNA, or a further amplified signal, may be required.

TABLE 1
The linear detection of UBQ5

| Total RNA | Hybridization Signal |
|---|---|
| 1 μg | 68.9 |
| 3 μg | 159.2 |
| 8 μg | 273.5 |

Table I shows the detection of UBQ5 from 1 μg, 3 μg and 8 μg of total RNA extracted from wild type Arabidopsis leaves. A strong linear relationship was observed between the signal and the amount of RNA sample used.

TABLE 2
A multiplex assay for detection of four genes

| | | Relative signal of |
|---|---|---|
| Gene | Relative signal of WT | WT.I |
| PRI | 0.00 | 111 |
| UBQ5 | 100 | 100.00 |
| PAD4 | 0.00 | 75 |
| PDF1.2 | 8.33 | 88 |

Total RNA was extracted from Arabidopsis leaves of wild type (XVI) and wild type with infection (WT.I) separately.

### Example 4

Detecting Gene Expression from In Vitro Transcripts, cRNA, or Quantitative PCR Products

To detect low copy genes using a microfluidics/fluorochrome system, linear amplification of the low copy genes may be necessary. In vitro transcription and quantitative PCR are two of the established approaches to amplify genes in the linear range.

In Vitro Transcription. Total RNA purified from samples is transcribed into cDNA using T7-poly-(dT) as primers by reverse transcriptase (Gibco BRL). The cDNA is transcribed back to cRNA (Ambion, Austin, Tex.). Biotin-UTP is incorporated into the cRNA during the synthesis. In general, all gene transcripts in total RNA are linearly amplified 50 to 100 times after in vitro transcription into cRNA. Thus, the level of low copy genes is enhanced proportionally to their original level and the enhanced level, allowing successful detection of rare genes by the technology of this invention.

Quantitative PCR. Rarely expressed genes are amplified by PCR from cDNA using their specific primers. Usually, amplification of low copy genes does not reach a plateau within 20 cycles of PCR, although the individual amplification curve of each gene is different. The amplification curve can be readily obtained by a quantitative PCR machine (Real-Time PCR, Perkin Elmer, Norwalk, Conn.). For labeling, either fluorescent (biotinylated) primers or fluorescent (biotinylated) deoxynucleotides are used in PCR reactions. The labeled PCR products are utilized for analysis by this technology.

### Example 5

Diagnostic Studies for Clinical Samples

After identification of genes for specific diseases, the method of the invention is used to detect normal or abnormal expression levels of disease genes in patient samples. The capture probe is chosen as a part of the gene that is identified as being associated with the disease. Selection of a capture probe involves choosing regions of the gene most conducive to an unambiguous identification of the transcripts. Regions that show little homology to other genes are most useful.

Any of the methods described herein can be used for the detection of the diagnostic nucleic acid. However, in the following example, detection at the RNA level is performed. In this specific approach, capture probes are anti-sense sequences of oligonucleotides coupled with microspheres, gene-representing targets are RNA transcripts, and reporters are anti-sense oligonucleotides corresponding to different segments of the target RNAs labeled with fluorescent dyes.

A unique sequence of 22 bases close to the 3′-end of a gene of interest is chosen as the capture probe oligonucleotide. The capture probe oligonucleotide is synthesized with 5′-amino uni-linker and then covalently linked to the carboxylated microspheres by the carbodiimide coupling procedure. Fluorescent labeled reporter oligonucleotides are designed and synthesized to hybridize to the RNA transcripts captured on the beads. The sequence of 22 bases adjacent to the oligonucleotides of capture probe is selected for this purpose. In order to increase sensitivity of the detection, two oligonucleotides adjacent to the capture probe sequence are selected, one upstream and the other downstream of the capture probe. A fluorescent dye is present at the 5′-end of the upstream reporter oligonucleotides and at the 3′-end of downstream reporter. The different positions of fluorescent dye in the two reporter oligonucleotides are chosen to minimize the steric hindrance to their hybridization to the target RNA.

Total RNA extracted from the clinical samples is fragmented and then denatured by incubation at 100° C. for 10 min. in a hybridization buffer of 1× TMAC. Microspheres coupled with capture probes and reporter oligonucleotides labeled with biotin (or fluorescent dyes) are then added to the denatured RNA and incubated at 55° C. for. 10 min. Target genes are hybridized selectively to their probes on microspheres and to their reporter oligonucleotides. Hence, complexes containing probe, reporter and target genes on microspheres are formed. The mixture is then subjected to analysis on a microfluidics analyzer.

This protocol is useful in a number of applications for quantitating or detecting disease-associated genes. Often a single nucleotide polymorphism (SNP) or a finite number of such SNPs is associated with a disease. Each known SNP is selected as the capture probe in the above example. Conditions for the protocol are selected such that only those genes with the polymorphism are hybridized. Taking the length and composition of the studied nucleic acid fragment into account, a hybridization temperature can be selected such that a single point mutation in a fragment can be discriminated from a perfectly matched sequence. Using preferred nucleic acid, fragments, such a temperature is typically between 35° C. and 75° C., preferably between 40° C. and 70° C., more preferably between 45° C. and 65° C., and most preferably about 50° C., 55° C., or 60° C. Because a number of different capture probes can be used in the same assay, a number of different polymorphisms can be identified simultaneously. Identification of such polymorphisms can greatly facilitate screening for genetic diseases. One example of such a disease associated gene is the human mannose binding protein (MBP) gene. MBP has four distinct structural alleles. Inheritance of any of the variant forms of MBP results in an immunologic defect. Another example is cystic fibrosis, in which a single nucleotide change causes severe disorders in children. Likewise, BRCA1 and BRCA2 alleles are associated with breast cancer. Thus, qualitative or quantitative detection of certain alleles is one particularly beneficial use of the technology of the present invention.

Quantitation of gene expression levels of a cancer-related gene product can be used to identify the stage of the disease. For example ovarian cancer marker genes, such as HE4 protease inhibitor, M₂ type pyruvate kinase, and mesothelin have been shown to be overexpressed in ovarian cancer, and screening for early detection of such over-expression is an important application of the present invention.

### Example 6

Disease Screening

An oligonucleotide capture probe specific for Ewing's sarcoma is linked to a first class of microbeads that can be identified by a first fluorescence identity. A rhabdomyosarcoma-specific capture probe is linked to second class of microbeads with a second fluorescence identity. A sample of DNA from a patients tumor is isolated and denatured, and the single-stranded nucleic acids are mixed with the substrate consisting of the mixed bead/capture probe combinations. Klenow fragment is added and strand extension incorporates nucleotides having a green fluorescent label. If beads of the first class ha e a green label, the tumor can be identified as a Ewing's sarcoma. If both classes of beads contain the green label, it is a mixed tumor. If only beads of the Blond class have a green label, the tumor is a rhabdomyosarcoma. The patient can then be treated consistent with the proper diagnosis. The same technique allows for the staging of many types of cancers by quantitating the amount of a specific target nucleic acid.

### Example 7

Candidate Gene Evaluation

Many protocols exist for profiling genome-wide gene expression. From any of such protocols, many candidate genes can be found to be associated with a specific trait, especially for quantitative traits (those that are associated with the combined actions or interactions of multiple genes). Accordingly, the methods of the invention can be used to monitor the candidate genes.

Genome wide gene expression is compared among several varieties of corn having high oil content, but otherwise having very different genetic backgrounds. From the study of various high oil corn varieties, a set of genes are identified that are suggested to be associated with high oil content. These genes are then evaluated and confirmed for their correlation with oil content.

Crosses in which one or both parents are high oil varieties are then conducted. Progeny of the cross are screened using the methods of the invention, to identify qualitatively whether a particular individual has the genes of interest. Likewise, the individual can be evaluated qualitatively to assess the degree of expression of one or more of the genes of interest. Oil content for each individual thus screened is assayed for confirmation of which candidate genes are most strongly correlated with oil content. The genes thus confirmed are used to screen progeny of subsequent crosses in the development of new varieties of high oil corn.

### Example 8

Expression Marker-Assisted Breeding

Markers or genes associated with specific desirable or undesirable traits are known and used in marker assisted breeding programs. It is particularly beneficial to be able to screen large numbers of markers and large numbers of candidate parental plants or progeny plants. The method of the invention allows high volume, multiplex screening for numerous markers from numerous individuals simultaneously. In accordance with this method, resistance to three different pathogens is screened in a large population of progeny from an open pollination cross involving parent plants hazing varying levels of resistance to at least one of the pathogens. Resistance to the first pathogen is a qualitative matter: plants carrying three different markers are resistant, and plants with any less than all three are not. Resistance to the second and third pathogens are quantitative due to variable expressivity of the associated genes: the higher expression levels of the relevant genes, the greater the plant's resistance to the pathogen.

A multiplex assay is designed providing capture probes specific to each of the five markers of interest. The capture probes are linked to five different classes of beads. All of the relevant markers are expressed genes, so RNA or cDNA techniques are appropriate. RNA is extracted from leaf tissue of 1000 different individual plants and hybridized in parallel reactions with the five different classes of beads. Each class of beads is analyzed for each sample using a microfluidics analyzer. For the three classes of beads corresponding to qualitative traits, qualitative measures of presence or absence of the target gene are recorded. For the two classes of beads corresponding to quantitative traits, quantitative measures of gene activity are recorded. Individuals showing activity of all of the qualitative genes and highest expression levels of the two quantitative traits are selected for further breeding steps. In procedures wherein no individuals have desirable results for all five measured genes, individuals having the most desirable, and fewest undesirable, results are selected for further breeding steps. In either case, progeny are screened to further select for homozygotes with high quantitative levels of expression of the quantitative traits.

Traits associated with the function of a single gene include: many disease resistance traits such as, for example, resistance to bacteria, viruses, fungi, nematodes, and insects; many herbicide resistance traits; many fruit or flower color traits; and various traits relating to male sterility. Likewise, traits associated with multiple genes or quantitative inheritance include: many disease resistance traits such as, for example, resistance to bacteria, viruses, fungi, nematodes, and insects; many yield or productivity traits; many fruit quality train; traits associated with tolerance to stresses such as heat, humidity, drought, salinity, and the like; traits associated with seedling emergence and the synchrony of flowering and/or fruiting.

### Example 9

Examination of the Effect of Chemical Compounds on Plants

Marker genes are detected by the invention to evaluate the effect of chemical compounds on crops. The screening process can be readily developed into a high throughput format. Large quantities of samples are able to be screened with a high speed unmatched by any conventional method. Each capture probe is complementary to a region of a marker gene. A number of capture probes are chosen and linked to specifically identifiable microbeads, such that analysis of all of the marker genes can be performed in a single assay.

For example, several chemical compounds are tested for their effect on root production and physiology. The effect of these compounds on plants is evaluated by the expression level of known genes involved in root formation, as well as genes known to be differentially expressed in roots. RNA is extracted from root tissue and is reverse transcribed to produce cDNA. The screening takes place as described above for second strand cDNA, and chemicals hating a pronounced effect on root-associated gene expression are selected for further study. As an alternative, the screening protocol is designed using capture probes to hybridize to mRNA of selected genes, and further employs labeled probes complementary to those sequences. Thus, in some embodiments of the present invention, in situ labeling of the target nucleic acid via strand extension may be replaced by hybridization of the target nucleic acid with an unbound labeled probe, forming a microbead/capture probe/target/labeled probe complex suitable for quantitative or qualitative analysis in a microfluidics analyzer. Such embodiments contemplate the use of any suitable labeled probe including, for example, probes incorporating radionuclides, dyes, fluorescers, and the like, as well as branched probes capable of further hybridization or interaction at one or more branches thereof with other labeled probes or signal enhancers. Accordingly, the sensitivity of detection using the method of the invention can be adjusted by using different labeling strategies and signal enhancers, depending on the relative abundance of the target and other factors affecting signal strength.

The screening by this method may also be suitable for biological systems such as a cell or cell culture, a tissue, an organ, an individual organism, a population of individuals of a single taxon, or a combination of cells, tissues, organs, or individuals of different taxa. The method is thus advantageous for screening plants, animals, fungi, or microorganisms. Any substance capable of affecting gene regulation or expression may be a suitable target for such a screening method, including, for example, organic substances, ions, minerals, vitamins, hormones, gases, viruses, bacteria, fungi, and the like.

### Example 10

Testing Sample Preparation for Microarrays

For experiments on gene chips, the sample that is to be analyzed must be pre-tested on special test chips. The pre-testing assures the quality of that sample and efficiency of labeling before it is applied to the more expensive analysis chips. Although the test chips are less expensive than the actual analysis chips each the test chip is still relatively costly. A great deal of time and money are saved if the test is carried out by the present invention instead of using test chips as suggested by gene chip suppliers.

The capture probes are selected as complementary to known genes that should be present in the test nucleic acid sample. For example, a variety of control genes selected from highly expressed groups, moderately expressed groups and rarely expressed groups are selected. The sample that will be analyzed is produced as follows: Total RNA or mRNA is isolated from two cell lines, HELA alone (control) and HELA that is expressing a BCR/ABL construct. Using an oligo dT T7 primer, ds cDNA copies are made from the RNA. The cDNA is then transcribed in the presence of biotinylated dUTPs to produce labeled cRNA. The cRNA is then used to hybridize to the chip. However, for use in the present invention, the cRNA is analyzed as follows:

The capture probes corresponding to different control messages are incubated with the cRNA at 55° C. for 10 min. Biotin-labeled target genes are then hybridized selectively to their probes on microspheres. Hence, complexes containing capture probes and labeled target genes on microspheres are formed. In the last step, PE conjugated streptavidin is added to the reaction and allowed to reach with the biotinylated cRNA. The mixture is subjected to analysis on a microfluidics analyzer. The sample is judged to be of high quality if the control genes are detected as expected. The quality of a cDNA library or other type of library can also be tested in this way.

### Example 11

Verification of Results from a Differential Display Experiment

The method can be used for verification and confirmation of results from differential display to minimize or eliminate the possibility of false data.

For example, if a differentially expressed gene is identified by differential display, the capture probe is selected to correspond to a region of the differentially expressed gene that is most conducive to an unambiguous result (a region with little homology to other known genes). Differential display is conducted to analyze a cell line, such as HELA cells, expressing a gene of interest (such as the BCR/ABL gene product). The assay is performed using the two nucleic acid samples: one from HELA alone, and one from HELA/BCR/ABL. Following the method of the invention, the level of the identified gene is then confirmed quickly and easily without the use of northern blots or the development of quantitative PCR. The RNA is extracted from each cell line (HELA, and HELA/BCR/ABL) and either directly added to the capture probe-substrate complex or the corresponding cDNA is added or the expression is detected at the level of second strand cDNA. Alternatively, if the gene of interest is a low copy gene, extra amplification via secondary labeling can be carried out, as discussed above. Microfluidics analysis of the microbeads from the reaction is then conducted, and differential expression of the relevant gene is unambiguously determined.

### Example 12

Testing the Efficiency of Promoters

The gene expression level regulated by different promoters is examined by this invention. It is useful for testing the efficiency of a novel promoter that has been identified in a plant, animal or microbe. The strength of a promoter or its differential expression in tissues is analyzed using the present invention. The capture probe is selected to be complementary to the gene product that is expressed by the promoter. The sample is the tissue or cell line in which the promoter activity is being tested.

Alternatively, a promoter that has been mutated or altered is analyzed using the present invention. The target probe is selected to be the gene product expressed by the promoter. A promoter construct expressed in a vector is analyzed in this way by using nucleic acids extracted from transformed cells to analyze expression controlled by the promoter. A promoter construct that has been transformed into a tissue, cell line or other type of cell is analyzed in the same way, using the nucleic acid from the tissue, cell line, or other type of cell as the tester nucleic acid.

RNA is extracted from each cell line or tissue and one of the following takes place: either the RNA is directly added to the capture probe-substrate or the corresponding cDNA is added or the expression is detected at the level of second strand cDNA, as discussed in prior Examples. Alternatively, if the gene of interest is a low copy gene, further signal amplification can be employed.

### Example 13

General Gene Expression Analysis in High Throughput Format

Currently, most laboratories in universities and institutions cannot afford expensive gene chip equipment and chips for gene expression analysis. The low cost of this invention enables these laboratories to study gene expression in any system. The present invention is particularly adaptable to high throughput analysis. Therefore, any type of gene expression analysis can be adapted for use according to the invention. For example, genes encoding any important molecular targets such as proteases, protein kinases, transcription factors and phosphatases can be screened in enormous samples using this technology.

### Example 14

Improving the Design of Oligonucleotide Probes

The efficiency of oligonucleotide probes in a hybridization experiment is tested by the invention. This is particularly important prior to the use of the probes. A particular probe, a probe variant, and probes corresponding to various parts of a gene of interest are tested for the quality of the signal. It is well understood that the quality of a probe can be quite variable depending on the cross-hybridization to “like” sequences. Therefore, the present invention can be used to select the best quality probe for a hybridization-type experiment. For example, if an experiment is intended to isolate the human homologue of a novel mouse gene, it is important to have a good quality probe. Such a probe is tested with the present invention by using each of the various candidate probes as capture probes and human RNA or DNA as the nucleic acid to be tested. The probe can be DNA or RNA. A number of different probes are chosen to be complementary to the novel mouse gene of interest. Each probe is linked to a unique solid support in a single assay. The different probes can be identified based on the identity of each solid support unit. The assay is performed as disclosed in one or more of the prior Examples herein. In a preferred embodiment, the hybridization stringency is altered either by changes in temperature or by changing the concentration or stringency or the TMAC buffer. Those probes that show a greatest affinity and specificity under different stringencies are then used for screening a human library to identify the human homologue of the novel mouse gene.

### Example 15

Screening Drugs

The effect of a drug library on the expression level of certain target genes is screened by this invention in a high throughput format. The capture probes are complementary to the target genes of interest in the drug screening protocol. A nucleic acid sample is isolated from the cells or tissue after exposure to the drug. In this way the effect of the drug on these genes is quantitated. The fact that the assay can easily be conducted in a high throughput format makes it particularly useful for screening libraries of candidate drugs.

### Example 16

Detection of Gene Transcripts Directly from Total RNA

A gene transcript is detected directly from a mixture including one or more nucleic acid molecules, such as total RNA, using capture probes coupled with microspheres. The nucleic acid molecules in the mixture includes, for example, mRNA, cRNA, viral RNA, synthetic RNA, cDNA, genomic DNA, viral DNA, plasmid DNA, synthetic DNA, a PCR product, or the like, or mixtures thereof, and preferably is derived from a plant, animal, virus or fungus. To detect gene expression at the RNA level, mRNA is the nucleic acid molecule.

In one assay method, the capture probes, which are attached to a solid surface, such as a microsphere or a bead, is the complimentary, or anti-sense, oligonucleotide molecule corresponding to the first region of a target nucleic acid molecule, such as an mRNA. Target nucleic acid molecule is selectively hybridized to its corresponding probe on a microsphere. Hybridization occurs on the 5′ end of the target nucleic acid molecule, the 3′ end of the target nucleic acid molecule, or anywhere in between,. Following the hybridization, at least one additional labeled probe having a sequence complimentary to another region of the target nucleic acid molecule is hybridized to the nucleic acid molecule. The labeled probe is labeled with any of the labels known to those of skill in the art, including, but not limited to, radiolabeles, biotin-avidin labels, and fluorescent labels. By hybridizing additional labeled probes to the same target nucleic acid molecule, the detection sensitivity is increased.

### Example 17

Detection of Reverse Transcribed cDNA

A gene transcript is detected directly from a nucleic acid molecule using capture probes coupled with microspheres. The nucleic acid is, for example, mRNA, cRNA, viral RNA, synthetic RNA, cDNA, genomic DNA, viral DNA, plasmid DNA, synthetic DNA, a PCR product, or the like, and is derived from a plant, animal, or fungus. To detect gene expression at the cDNA level, cDNA is the nucleic acid molecule.

In one assay method, gene expression is analyzed from reverse transcribed cDNA. The sensitivity of the assay is enhanced by incorporation of a label into the newly synthesized cDNA. One example of a label is biotinylated deoxynucleotide. In this assay, the capture probes are sense sequences of oligonucleotides complimentary to cDNA. Target genes located on the 5′ end, the 3′ end, or anywhere in between, of the cDNA molecules selectively hybridize to the capture probes coupled with the microspheres. Additionally, strand extension using the single-stranded region of the captured cDNA is carried out using the captured target cDNA as a template. During DNA synthesis, labeled or modified nucleotides are incorporated into the second strand cDNA.

### Example 18

Detection of Second Strand cDNA

A gene transcript si detected directly from a nucleic acid molecule using capture probes coupled with microspheres. The nucleic acid is, for example, mRNA, cRNA, viral RNA, synthetic RNA, cDNA, genomic DNA, viral DNA, plasmid DNA, synthetic DNA, a PCR product, or the like, and preferably is derived from a plant, animal, or fungus.

In one assay method, gene expression is detected by second strand cDNA extension. The sensitivity of the assay is enhanced by incorporation of a label into the newly synthesized cDNA. One example of a label is biotinylated deoxynucleotide. In the assay, the capture probes are sense sequences of oligonucleotides complimentary to cDNA. Target genes located on the 5′ end, middle, or 3′ end of the cDNAs selectively hybridize to the capture probes coupled with the microspheres. Detection of second strand cDNA employs a method similar to detecting gene expression from cDNA, but includes the additional step of extending the second strand cDNA coupled to the microsphere. Then, a DNA polymerase, such as E. coli DNA polymerase I, is used to extend the second strand cDNA, using the capture probe as a primer and the first strand cDNA as template. The extension includes nucleotides having a label adapted to enhance the sensitivity of detection of the extension.

### CONCLUSION

Thus, it will be appreciated that certain methods of analysis of a nucleci acid sample has been disclosed.

One skilled in the art would readily appreciate that the present invention is well adapted to carry out the objects and obtain the ends and advantages mentioned, as well as those inherent therein. The methods and procedures described herein are presently representative of preferred embodiments and are exemplary and are not intended as limitations on the scope of the invention. Changes therein and other uses will occur to those skilled in the art which are encompassed within the spirit of the invention and are defined by t*e scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are within the following claims.

What is claimed is:

1. A method of analysis of a nucleic acid sample, comprising the steps of:
(a) providing a substrate comprising a solid support and a capture probe linked thereto, the capture probe having a sequence complementary to a first segment of a sequence of a single-stranded target nucleic acid;
(b) contacting the substrate with a nucleic acid sample, under conditions suitable for hybridization between the capture probe and the target nucleic acid, wherein upon the hybridization at least a second segment of the sequence of the target nucleic acid remains single stranded;
(c) exposing the substrate to conditions suitable for complementing at least a second segment of the target nucleic acid, wherein the complementing nucleic acid comprises nucleotides having a label capable of enhancing sensitivity of detection of the complementing nucleic acid; and
analyzing the label to determine presence or absence of the target nucleic acid in the nucleic acid sample.

2. A method of claim 1 wherein in step (c) the substrate is exposed to conditions suitable for polymerizing an extension complementary to at least a second segment of the target nucleic acid, wherein the extension comprises nucleotides having a label capable of enhancing sensitivity of detection of the extension

3. The method of claim 1, wherein in step (c) the substrate is exposed to conditions suitable for hybridization with a probe nucleic acid comprising nucleotides having a label capable of enhancing sensitivity of detection of the probe nucleic acid, which is complementary to part or all of at least a second segment of the target nucleic acid.

4. The method of any one of claims 1 to 3, wherein the solid support is selected from the group consisting of a microbead, a chromatography bead, an affinity bead, a gene chip, a membrane, a microtiter plate, a glass plate, and a plastic plate.

5. The method of claim 4, wherein the solid support is a fluorescent microbead.

6. The method of claim 5, wherein the microbead comprises a fluorochrome.

7. The method of claim 6, wherein the microbead comprises at least two different fluorochromes, wherein the different fluorochromes emit fluorescence at different wavelengths to indicate a fluorochrome identity of the microbead.

8. The method of claim 7, wherein the substrate comprises a plurality of microbeads of at least two different classes, wherein the classes are based on fluorochrome identities of the microbeads within each class, and wherein the different classes of microbeads correspond to different target nucleic acids.

9. The method of claim 8, further comprising the steps of:
detecting the fluorescence of each of the different fluorochromes to determine the fluorochrome identity of the microbead; and
correlating the analyzed label with the fluorochrome identity of the microbead.

10. The method of any one of claims 1 to 9, wherein the substrate comprises a plurality of species of capture probes, and wherein probes within each of the species have a sequence distinct from the probes of every other of the plurality of species.

11. The method of claim 10, wherein at least two of the plurality of species of capture probes correspond to different segments of a single target nucleic acid.

12. The method of claim 10, wherein the plurality of species of capture probes correspond to different target nucleic acids.

13. The method of any one of claims 1 to 12, wherein the substrate comprises more than 10 species of capture probes.

14. The method of any one of claims 10 to 13, wherein the solid support is selected from the group consisting of a gene chip, a membrane, a glass plate, and a plastic plate, wherein each of the species of capture probes is linked to a discrete region of the solid support.

15. The method of any one of claims 10 to 13, wherein the solid support is selected from the group consisting of a gene chip, a membrane, a glass plate, and a plastic plate, wherein each of a plurality of discrete regions of the solid support has liked thereto probes whose species is determined.

16. The method of any one of claims 10 to 13, wherein the substrate comprises a plurality of solid support units, wherein the solid support units are selected from the group consisting of a microbead, a chromatography bead, an affinity bead, a fluorescent bead, and a radiolabeled bead.

17. The method of claim 16, wherein each solid support unit has linked thereto only probes of one of the species.

18. The method of claim 16, wherein each solid support unit has liked thereto probes of whose species is determined.

19. The method of any one of claims 10 to 18, comprising the additional steps of:
identifying solid support regions or units indicative of presence of the target nucleic acid, based on the analyzing step;
determining the species of all of capture probes linked to solid support regions or emits of the identifying step;
providing a second substrate, the second substrate comprising the probe species of the determining step, wherein the probe species are distinguishable from each other based on a discrete position of each species on a solid support comprising a plurality of the species, or based on presence of only a single species on each of a plurality of solid support units;
contacting the second substrate with the nucleic acid sample, under conditions suitable for hybridization between a probe species and the target nucleic acid, wherein upon the hybridization, a second segment of the sequence of the target nucleic acid remains single stranded;
exposing the substrate to conditions suitable for polymerizing an extension complementary to the second segment of the target nucleic acid, wherein the extension comprises nucleotides having a label adapted to enhance sensitivity of detection of the extension; and
analyzing the label to identify a probe species hybridized to the target nucleic acid.

20. The method of any one of claims 1 to 19 wherein the capture probe is complementary to a region within approximately between 1000 and 600 bases from the 3′-end of the target nucleic acid.

21. The method of any one of claims 1 to 20 wherein the capture probe is composed of between 15 and 150 nucleotides.

22. The method of claim 21 wherein the capture probe is composed of between 20 and 60 nucleotides.

23. The method of claim 22 wherein the capture probe is composed of between 22 and 25 bases.

24. The method of any one of claims 1 to 23, wherein the region of complementation within the capture probe is unique amongst all the genes in the sample to be tested.

25. The method of any one of claims 1 to 24, wherein the nucleic acid sample is derived from a plant, animal, fungus or virus.

26. The method of any one of claims 1 to 25, wherein the target nucleic acid is selected from the group consisting of mRNA, cRNA, viral RNA, synthetic RNA, cDNA, genomic DNA, viral DNA, plasmid DNA, synthetic DNA, and a PCR product.

27. The method of claim 26, wherein the target nucleic acid is an mRNA.

28. The method of claim 26, wherein the target nucleic acid is a cDNA.

29. The method of any one of claims 1 to 28, wherein the target nucleic acid is derived from an organism and is associated with a specific phenotype or trait of the organism.

30. The method of any one of claims 1 to 29, wherein the extension is polymerized by an enzyme selected from the group consisting of a reverse transcriptase, a DNA polymerase, an RNA polymerase, and Klenow fragment, or by a mutant form of any member of the group.

31. A method according to any one of claims 1 to 30, wherein the ratio of labeled vs unlabeled nucleotides in the polymerization process is between 1:7 and 1:2, preferably between 1:5 and 1:2 and most preferably is 1:3.

32. A method according to any one of claims 1 to 30, wherein the complementing nucleic acid contains incorporated therein between about one modified or labeled nucleotide of every 10 to 50, preferably every 15 to 35, more preferably every 20 to 25 nucleotides.

33. A method according to claim 3 wherein the number of labeled probe molecules is in a range of between 1 and 10, preferably of between 1 and 5 and most preferably of between 1 and 3 probe nucleic acid molecules.

34. The method of any one of claims 1 to 33, wherein the label is selected from the group consisting of radionuclides, fluorescers, chemiluminescers, dyes, enzymes, enzyme substrates, enzyme cofactors, enzyme inhibitors, enzyme subunits, antigens, ligands, and metal ions.

35. The method of claim 34, wherein the label is selected from the group consisting of xanthine dyes, rhodamine dyes, naphthylamines, benzoxadiazoles, stilbenes, pyrenes, acridines, Cyanine 3, Cyanine 5, phycoerythrin, Alexa 532, fluorescein, TAMRA, tetramethyl rhodamine, fluorescent nucleotides, digoxigenin, and biotin.

36. The method of any one of claims 1 to 35, wherein the analyzing step comprises a quantitation of the label associated with the target nucleic acid.

37. The method of claim 36, wherein the analyzing step comprises a quantitation of the label associated with the target nucleic acid.

38. The method of any one of claims 1 to 37, wherein the microbead is sorted based on its fluorochrome identity.

39. The method of any one of claims 1 to 38, wherein the analysis is used to identify a single nucleotide polymorphism in the target nucleic acid.

40. A method of screening for changes in the expression or regulation of a target nucleic acid in a biological system, comprising the steps of:
(a) treating the biological system with a substance; or subjecting the biological system to changed environmental conditions;
(b) extracting a nucleic acid sample from the biological system;
(c) providing a substrate comprising a solid support and a capture probe linked thereto, the capture probe having a sequence complementary to a first segment of a sequence of a single-stranded target nucleic acid;
(d) contacting the substrate with the nucleic acid sample extracted from the biological system, under conditions suitable for hybridization between the capture probe and the target nucleic acid, wherein upon the hybridization a second segment of the sequence of the target nucleic acid remains single stranded;
(e) exposing the substrate to conditions suitable for complementing at least a second segment of the target nucleic acid, wherein the complementing nucleic acid comprises nucleotides having a label capable of enhancing sensitivity of detection of the complementing nucleic acid and wherein the complementation is preferably achieved by polymerizing an extension complementary to the second segment of the target nucleic acid, wherein the extension comprises nucleotides having a label capable of enhancing sensitivity of detection of the extension;
(f) analyzing the label to determine presence or absence of the target nucleic acid in the nucleic acid sample; and
(g) determining changes in the expression or regulation of the target nucleic acid in the biological system.

41. The method of claim 40, wherein the biological system is selected-from the group consisting of a cell or cell culture, a tissue, an organ, an individual organism, a population of individuals of a single taxon, and a combination of cells, tissues, organs, or individuals of different taxa.

42. The method of claim 41, wherein the system comprises a plant, an animal, a fungus, a virus or a part of a plant, animal, virus or fungus.

43. The method of claim 40, wherein the substance comprises one or more components selected from the group consisting of an organic substance, an ion, a mineral, a vitamin, a hormone, a gas, a virus, a bacterium, and a fungus.

44. The method of any one of claims 40 to 43, wherein the analyzing step comprises a quantitation of the label associated with the target nucleic acid.

45. The method of any one of claims 40 to 44, wherein the solid support is a microbead.

46. The method of claim 45, wherein the microbead comprises at least two different fluorochromes, wherein the different fluorochromes emit fluorescence at different wavelengths to indicate a fluorochrome identity of the microbead.

47. The method of claim 46, further comprising a steps of:
detecting the fluorescence of each of the different fluorochromes to determine the fluorochrome identity of the microbead; and
correlating the analyzed label with the fluorochrome identity of the microbead.

48. The method of any one of claims 40 to 47, wherein the target nucleic acid is selected from the group consisting of mRNA, cRNA, viral RNA, synthetic RNA, cDNA, genomic DNA, viral DNA, plasmid DNA, synthetic DNA, and a PCR product.

49. The method of any one of claims 40 to 48, wherein the target nucleic acid is derived from an organism and is associated with a specific phenotype or trait of the organism.

50. A system of gene expression analysis, the system comprising a microbead having at least two different fluorochromes, the system further comprising at least one capture probe linked to the microbead, the capture probe having a sequence complementary to a first segment of a sequence of a target nucleic acid, the system also comprising a labeled probe complementary to at least a second segment of the sequence of the target nucleic acid, wherein the labeled probe comprises a label capable of enhancing sensitivity of detection thereof.

51. The system of claim 50 wherein the labeled probe is a product of nucleic acid polymerization within the complex, using the second segment as a template therefor.

52. The system of claim 50, wherein the labeled probe comprises a first region complementary to the second segment of the target nucleic acid and a second region capable of interacting with a signal enhancer.

53. The system of claim 52, wherein the second region is branched in structure, having a plurality of ends, and wherein at least two of the ends are capable of interacting with a signal enhancer.

54. The system of claims 52 or 53, wherein the signal enhancer is selected from the group consisting of a labeled probe, radionuclides, fluorescers, chemiluminescers, dyes, enzymes, enzyme substrates, enzyme cofactors, enzyme inhibitors, enzyme subunits, antigens, ligands, and metal ions.

55. A diagnostic kit suitable for diagnosis of a particular physiological state of an organism, comprising a solid support and a capture probe linked to the solid support, wherein the capture probe is complementary to a first segment of a target nucleic acid associated with the physiological state.

56. The kit of claim 55, further comprising a probe capable of hybridizing to a second segment of the target nucleic acid.

57. The kit of claim 56, wherein the probe comprises a label capable of enhancing sensitivity of detection thereof.

58. The kit of claim 55, further comprising components necessary for extension of a probe complementary to a second segment of the target nucleic acid.

59. A method for marker assisted breeding comprising the steps of:
providing a substrate comprising a solid support and a capture probe linked thereto, the capture probe having a sequence complementary to a first segment of a sequence of a target nucleic acid, wherein the target nucleic acid is correlated with a trait of interest in a breeding program;
contacting the substrate with a nucleic acid sample from an individual or population in the breeding program, under conditions suitable for hybridization between the capture probe and the target nucleic acid;
probing a second segment of the target nucleic acid to detect presence or absence of the target nucleic acid; and
determining desirability of the individual or population for the breeding program, based on the presence or absence of the target nucleic acid, whereby the individual is used for marker assisted breeding.

60. The method of claim 59, wherein the probing step comprises polymerization of a probe using the second segment as a template therefor.

61. The method of claim 59, wherein the solid support comprises a microbead having at least two different fluorochromes.

62. The method of any one of claims 59 to 61, wherein the trait is correlated with a plurality of target nucleic acids, and wherein the substrate comprises capture probes complementary to at least two of the target nucleic acids.

63. The method of any one of claims 59 to 62, wherein the method is used to screen candidates for breeding.

64. The method of any one of claims 59 to 63, wherein the method is used to screen progeny of the breeding program for end use or for subsequent breeding steps.

65. A method of determining effectiveness of a capture probe, comprising the steps of:
providing a substrate comprising a solid support and a capture probe linked thereto, the capture probe having a sequence complementary to a first segment of a sequence of a single-stranded target nucleic acid;
contacting the substrate with a nucleic acid sample, under conditions suitable for hybridization between the capture probe and the target nucleic acid, wherein upon the hybridization at least a second segment of the sequence of the target nucleic acid remains single stranded;
exposing the substrate to conditions suitable for polymerizing an extension complementary to the second segment of the target nucleic acid, wherein the extension comprises nucleotides having a label capable of enhancing sensitivity of detection of the extension; and
analyzing the label quantitatively to determine effectiveness of the capture probe in capturing the target nucleic acid.

66. A method of analysis of a nucleic acid sample, comprising the steps of providing a substrate comprising a solid support and a capture probe linked thereto; providing a single-stranded target nucleic acid sample, comprising at least a first segment, a second segment, and a third segment, wherein the capture probe has a sequence complementary to a portion of one of the segments;
contacting the substrate with the nucleic acid sample, under conditions suitable for hybridization between the capture probe and the target nucleic acid, wherein upon the hybridization at least two of the segments of the nucleic acid sample remain single stranded,
contacting the substrate with at least one labeled probe, under conditions suitable for hybridization between the labeled probe and a portion of a single stranded segment of the nucleic acid sample, wherein the labeled probe comprises a nucleic acid sequence complementary to at least a portion of the single stranded segment of the nucleic acid sample; and
analyzing the label to determine presence or absence of the target nucleic acid in the nucleic acid sample.
